(19) United States
(12) Patent Application Publication
(10) Pub. No.: US 2021/0259736 A1
(43) Pub. Date: Aug. 26, 2021

(54) IMPLANTATION NEEDLE AND KIT

(71) Applicant: Roche Diabetes Care, Inc., Indianapolis, IN (US)

(72) Inventors: Gernot Hochmuth, Mannheim (DE); Alexander Steck, Hirschberg (DE)

(21) Appl. No.: 17/243,160

(22) Filed: Apr. 28, 2021

Related U.S. Application Data
(63) Continuation of application No. PCT/EP2019/078915, filed on Oct. 23, 2019.

(30) Foreign Application Priority Data
Oct. 30, 2018 (EP) 18 203 395.1

(51) Int. Cl. A61B 17/34; A61M 37/00

(52) U.S. Cl. CPC A61B 17/3468; A61B 17/3415; A61B 2560/063; A61M 37/0069

(57) ABSTRACT

An implantation needle has an implantable element receiving portion, a capsule receiving portion, and a tip portion. The capsule receiving portion has a fixing element such as a mechanical barrier or an adhesive surface. A kit is also provided that includes the implantation needle with an implantable element disposed in the implantable element receiving portion and a capsule disposed in the capsule receiving portion. The instant disclosure also relates to a method for administering an implantable element and a capsule to a subject. The method includes inserting an implantation needle of the implantation kit into a tissue of a subject and discharging the implantable element and capsule into the patient's tissue.

### RELATED APPLICATIONS

This application is a continuation of PCT/EP2019/078915, filed Oct. 23, 2019, which claims priority to EP 18 203 395.1, filed Oct. 30, 2018, the entire disclosures of both of which are hereby incorporated herein by reference.

### BACKGROUND

This disclosure relates to an implantation needle comprising an implantable element receiving portion, a capsule receiving portion, and a tip portion, wherein the capsule receiving portion comprises at least one fixing element selected from the group consisting of a mechanical barrier and an adhesive surface. This disclosure further relates to an implantation kit comprising the implantation needle of this disclosure, an implantable element comprised in the implantable element receiving portion of said implantation needle, and a capsule comprised in the capsule receiving portion of said implantation needle. Furthermore, this disclosure relates to a method for administering an implantable element and a capsule to a subject, said method comprising inserting an implantation needle of an implantation kit according to this disclosure into a tissue of the subject and discharging said implantable element and said capsule into said tissue; and to a use of an implantation needle according to this disclosure and/or of an implantation kit according to this disclosure for administering an implantable element and a capsule to a subject.

Immunosuppressive pharmaceutical compounds have a very broad spectrum of medical uses, from self-medication in treatment of allergic coryza to suppression of rejection in graft recipients. Immunosuppressive compounds are often provided as immediate release formulations, however, in some applications sustained-release formulations may be preferred. E.g., in the field of ophthalmology, corticosteroids have been used for the treatment of macular edema. As an intravitreal delivery system, a pen-like instrument was proposed, delivering a dose of a small, biodegradable and sustained-release formulation of dexamethasone (Meyer et al. (2012), Retina 32:2133; Fialho & da Silva Cunha (2005), Drug Delivery 12(2): 109).

For inserting implantable elements, e.g., implantable sensors, under the skin of a subject, a variety of implantation needles are known, e.g., closed cannulas with a V-bevel, oval-shaped slotted cannulas with a V-bevel, or peel catheters, i.e., a cannula tube divided into two with a V-bevel which is then opened in the skin and removed in separate parts; e.g., as described in WO 2015/128263 A1, DE 10 2011 112 021 A1, DE 102 24 101 A1, WO 99/53991 A1, U.S. Publication No. 2010/0324579, U.S. Pat. Nos. 3,064,651, 3,448,740, WO 2005/044116, and U.S. Pat. No. 4,490,139.

A common problem with implantation of implantable elements is that the implantable elements may cause a defense reaction of the body of a subject to occur, which may lead to inflammation, scarring, and other immune responses to the non-self material (i.e., material identified by the immune system as foreign to the body) of the implantable element. For this reason, use of pharmaceutical compounds improving tolerance has been proposed, in particular topical administration at the site of implantation (cf., e.g., CA 2 664 426; WO 2006/055008). Moreover, implantable elements, in particular implantable sensors like continuous glucose measurement (CGM) sensors, have been covered with membranes comprising pharmaceutical compounds improving tolerance to suppress local immune reactions (cf., e.g., Reichert et al. (2016), Acta Biomaterialia, 30, 106-115). Nonetheless, immune reactions and rejection of implantable elements cannot always be avoided. Moreover, manufacturing of coated implantable elements is rather complex and, therefore, cost-intensive.

WO 2006/023315 discloses a brachytherapy needle assembly for simultaneously dispensing an effective amount of medicine along with radioactive seeds.

U.S. Publication No. 2009/0131908 discloses needles for delivering drug depots to a site beneath the skin. The needle comprises a drug depot chamber.

WO 2011/106502 discloses a method for delivering drugs to desired locations within a mammal, in particular to implant a guide wire and a drug eluting implant.

U.S. Publication No. 2007/0243228 discloses drug depot implant designs for delivery of therapeutic agents.

### SUMMARY

This disclosure teaches improved means and methods for preventing immune reactions to implantable elements, avoiding at least in part the drawbacks of the prior art.

Accordingly, this disclosure relates to an implantation needle comprising an implantable element receiving portion, a capsule receiving portion, and a tip portion, wherein the capsule receiving portion comprises at least one fixing element selected from the group consisting of a mechanical barrier and an adhesive surface.

As used in the following, the terms “have,” “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B,” “A comprises B” and “A includes B” may both refer to a situation in which, besides B, no other element is present in A (i.e., a situation in which A solely and exclusively consists of B) and to a situation in which, besides B, one or more further elements are present in entity A, such as element C, elements C and D or even further elements.

It shall also be understood for purposes of this disclosure and appended claims that, regardless of whether the phrases “one or more” or “at least one” precede an element or feature appearing in this disclosure or claims, such element or feature shall not receive a singular interpretation unless it is made explicit herein. By way of non-limiting example, the terms “needle,” “capsule,” and “fixing element,” to name just a few, should be interpreted wherever they appear in this disclosure and claims to mean “at least one” or “one or more” regardless of whether they are introduced with the expressions “at least one” or “one or more.” All other terms used herein should be similarly interpreted unless it is made explicit that a singular interpretation is intended.

Further, as used in the following, the terms “preferably,” “more preferably,” “most preferably,” “particularly,” “more particularly,” “specifically,” “more specifically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment” or similar expressions are intended to be optional features, without any restriction regarding further embodiments of the invention, without any restrictions regarding the scope of the invention and without any restriction regarding the possibility of combining the features introduced in such way with other optional or non-optional features of the invention. If not otherwise indicated, the term “about” relates to the indicated value with the commonly accepted technical precision in the relevant field, preferably relates to the indicated value±20%, more preferably ±10%, most preferably ±5%. Further, the term “essentially” indicates that deviations having influence on the indicated result or use are absent, i.e., potential deviations do not cause the indicated result to deviate by more than ±20%, more preferably ±10%, most preferably ±5%.

The term “subject,” as used herein, relates to a mammal, for example to a mouse, a rat, a pig, a guinea pig and/or a human subject, in particular to a human subject.

The term “biocompatible,” as used herein, relates to the property of a compound or device, in particular an implantable element as specified elsewhere herein, of not being deleterious to a living system, in an embodiment a subject, contacted therewith. In an embodiment, the term relates to the ability of a compound or device, in particular an implantable element as specified elsewhere herein, to be in contact with a living system, in an embodiment a subject, producing only a local response, in an embodiment without producing a generalized immune response, in a further embodiment without producing an adverse effect.

The term “biodegradable,” as used herein, relates to the property of a compound of being degradable by a living system, in particular a subject. In an embodiment, the term relates to biodegradability according to the IUPAC definition, i.e., to a compound susceptible to degradation by biological activity, with the degradation accompanied by a lowering of its molecular mass. In an embodiment, said biodegradability is biodegradability within a subject, in an embodiment biodegradability is after subcutaneous implantation into a subject. In an embodiment, biodegradability requires a degradation half-life of a compound of at most 30 days after subcutaneous implantation in a mammal, in an embodiment a mouse. Methods for determining the half-life of a biodegradable compound are known in the art; e.g., for poly(D,L-lactide-co-glycolide) from Farahani et al. (2005), Iranian Polymer Journal 14 (8): 753-763. In an embodiment, said degradation half-life is determined after subcutaneous implantation of a spherical mass of 0.1 g of the compound, in an embodiment into a mouse.

The term “implantation needle,” as used herein, relates to a device of generally elongated form, adapted to pierce a solid or semisolid surface, in particular the skin of a subject, and adapted for implanting an implantable element and a capsule under the skin of a subject, said implantation needle comprising an implantable element receiving portion, a capsule receiving portion, and a tip portion. In an embodiment, the implantable element receiving portion is the proximal receiving portion, the terms “proximal” and “distal” being used herein relative to an operator of the implantation needle or implantation kit; thus, e.g., with regards to the implantation needle, the tip portion is the most distal portion. Thus, in an embodiment, the implantation needle comprises the described elements in the order implantable element receiving portion, capsule receiving portion, and tip portion. Thus, in an embodiment, in the implantation needle, the capsule receiving portion is distal to the implantable element receiving portion. In an embodiment, the implantation needle comprises or consists of a rigid or semi-rigid material, in an embodiment a metal, a plastic, or a ceramic, in a further embodiment a metal. In an embodiment, the implantation needle comprises or consists of steel, in an embodiment stainless steel. In an embodiment, the implantation needle further comprises a connecting portion, extending the distance between (i) the tip portion and (ii) the implantable element receiving portion and/or the capsule receiving portion, as specified herein below. As used herein, the sections of the implantation needle comprising the implantable element receiving portion, the capsule receiving portion, and, if present, the connecting portion, are together also referred to as “implantation needle main body.”

As used herein, the term “implantable element receiving portion” relates to a section of the implantation needle adapted for receiving an implantable element as specified elsewhere herein. Thus, in an embodiment, the implantable element receiving portion has dimensions suitable for receiving an implantable element; in an embodiment, the implantable element receiving portion has a largest inner diameter of from 0.25 mm to 1.5 mm, in a further embodiment of from 0.5 mm to 1 mm, in a further embodiment of about 0.65 mm, in a further embodiment of 0.67 mm. In an embodiment, the implantable element receiving portion has a length of from 1 mm to 15 mm, in an embodiment of from 5 mm to 12 mm.

As used herein, the term “capsule receiving portion,” relates to a section of the implantation needle adapted for receiving a capsule as specified elsewhere herein and comprising at least one fixing element as specified elsewhere herein. Thus, in an embodiment, the capsule receiving portion has dimensions suitable for receiving a capsule; in an embodiment, the capsule receiving portion has a largest inner diameter of from 0.25 mm to 1.5 mm, in a further embodiment of from 0.5 to 1 mm, in a further embodiment of about 0.65 mm, in a further embodiment of 0.67 mm. In an embodiment, the capsule receiving portion has a length of from 1 to 10 mm, in an embodiment of from 1.5 to 3 mm, in a further embodiment 1.8 mm.

The term “tip portion,” as used herein, relates to a pointed and/or sharpened distal section of the implantation needle, adapted to pierce a solid or semisolid surface, in particular the skin of a subject. As is understood by the skilled person, the pointed and/or sharpened section of the implantation needle typically is the distal section of the implantation needle. In its proximal section, the tip portion is adapted to provide a transition of the distal end of the capsule receiving portion or of the implantable element receiving portion to a pointed and/or sharpened section of the tip portion, i.e., the tip. Thus, the tip portion may in particular comprise a slant surface, in particular as specified elsewhere herein.

The term “connecting portion,” as used herein, relates to a portion of the implantation needle having essentially the same features as the implantable element receiving portion and/or the capsule receiving portion, however, lacking a fixing element. Thus, in an embodiment, a connecting portion may intervene between the distal end of the capsule receiving portion and the proximal end of the tip portion, thus providing an overall longer needle, permitting deeper insertion of the implantable element and the capsule into the body of a subject.

In an embodiment, the implantable element receiving portion is adapted to receive the implantable element as specified elsewhere herein. In a further embodiment, the implantable element receiving portion is adapted or further adapted to receive the capsule as specified elsewhere herein. Also in an embodiment, the capsule receiving portion is adapted to receive the capsule as specified elsewhere herein. In a further embodiment, the capsule receiving portion is adapted or further adapted to receive the implantable element as specified elsewhere herein.

The implantation needle comprises at least one inner lumen completely or partially enclosed laterally by the rigid or semi-rigid material of the needle. In an embodiment, the implantation needle is an open implantation needle, e.g., a slotted implantation needle as specified herein below, in a further embodiment, the implantation needle is a closed implantation needle, i.e., the rigid or semi-rigid material completely encloses the inner lumen along the elongated axis. In an embodiment, a cross-section of the inner lumen of the implantation needle is essentially round, in particular circular or oval, is rectangular, is V-shaped, or is U-shaped, in a further embodiment is essentially round. In an embodiment, a cross-section of the inner lumen has the same shape over at least ½, in a further embodiment ⅔ of needle length, in an embodiment has essentially the same shape from the proximal end of the tip portion to the proximal end of the implantable element receiving portion and/or the proximal end of the capsule receiving portion. In an embodiment, a cross-section of the inner lumen of the implantation needle has an essentially round shape; in such case, in an embodiment, the largest inner diameter of the implantation needle is of from 0.25 mm to 1.5 mm, in a further embodiment of from 0.5 to 1 mm, in a further embodiment of about 0.65 mm, in a further embodiment of 0.67 mm. In an embodiment, the largest inner diameter is essentially constant over at least ½, in a further embodiment ⅔ of implantation needle length, in an embodiment is constant at least from the proximal end of the tip portion to the proximal end of the implantable element receiving portion and/or the proximal end of the capsule receiving portion. In an embodiment, the cross-section of the inner lumen of the implantation needle has a U or V shape; in such case, the above values for a largest inner diameter apply to the largest distance of the lateral walls mutatis mutandis. As will be understood by the skilled person, also mixed open/closed embodiments are envisaged, e.g., implantation needles closed at the implantable element receiving portion, but open at the capsule receiving portion and/or at the tip portion. Thus, in an embodiment, the implantation needle is closed, in a further embodiment the implantation needle is slotted, wherein the implantation needle may be slotted over the whole length of the implantation needle, or may be slotted over only a part of its length, in an embodiment including the distal portion or portions. Thus, in an embodiment, the implantation needle is a slotted cylindrical implantation needle, a U-shaped implantation needle, or a slotted implantation needle with a rectangular cross-section. In an embodiment, the implantation needle is an implantation needle essentially as disclosed by WO 2018/166963 A1.

Thus, in an embodiment, the implantation needle comprises an implantable element receiving portion and a capsule receiving portion and a slant tip portion. The slant tip portion, in an embodiment, further comprises: a first slant surface contiguous to a first outer peripheral surface of the implantation needle main body, wherein the first slant surface is provided as a first non-cutting edge; a second slant surface contiguous to a second outer peripheral surface of the hollow implantation needle main body, wherein the second slant surface is provided as a second non-cutting edge; and a pair of sharpened surfaces symmetric with respect to an edge point and a longitudinal axis of the implantation needle main body, wherein the sharpened surfaces are both provided with a cutting edge. The first slant surface comprises a first flank, and the second slant surface comprises a second flank. The first flank is provided at a first distance from the edge point, and the second flank is provided at a second distance from the edge point which is different from the first distance.

As indicated above, the implantation needle main body may be an open implantation needle main body. In other words, in an embodiment, the implantation needle main body does not have a closed tube shape. The opening in the side of the implantation needle main body may extend along the entire length of the implantation needle main body. Alternatively, the opening in the side of the implantation needle main body may only extend along part of the length of the implantation needle main body. In case the opening extends only along part of the length of the implantation needle main body, the opening, in an embodiment, extends to a distal end of the implantation needle main body, providing an opening towards the slant tip portion. In an embodiment, the opening in the side of the implantation needle main body may be formed symmetric with respect to the longitudinal axis of the implantation needle main body. The opening in the side of the implantation needle main body may be provided as a slot opening. Thereby, a slotted implantation needle main body may be provided. Inner edges formed in the range of the opening in the side of the implantation needle main body may be provided as non-cutting edges.

In the following it is described how an implantation needle as specified above can be manufactured. The method may comprise a) punching a flat metal strip or sheet so as to give rise to a flat sheet of a desired shape suitable for later bending the sheet so as to give rise to the shape of the implantation needle. In a second step b) the sheet may then be subjected to embossing of the “dull” non-cutting edges in the portion of the sheet. Then, in a step c) the implantation needle main body and the slant surfaces of the tip portion may be bent and the tip portion may be embossed and punched out so as to give rise to the implantation needle of this disclosure. As an alternative, etching methods can be used to create a sharp tip of the implantation needle. In a further step d), a fixing element, in particular a tongue, may be punched out. As will be understood by the skilled person, step d) may also be performed concomitantly to step a). The method for manufacturing the implantation needle may comprise producing at least the first and second flanks by at least one of a punch-bent process or an etching process combined with a bent process. The punch-bent process combining punching and bending the material used for manufacturing the implantation needle are combined for producing at least one of the flanks. Such punch-bent process may be used for manufacturing the implantation needle main body as well.

The capsule receiving portion of the implantation needle comprises at least one fixing element. The term “fixing element” (also referred to herein as a “capsule stopper”) is understood by the skilled person to relate to any element of the implantation needle adapted to restrain a capsule or an implantable element comprised in the capsule receiving section from moving in the direction of the tip portion in the absence of external force. In an embodiment, the at least one fixing element is a mechanical barrier and/or an adhesive surface. In an embodiment, the at least one fixing element is located proximal to the tip portion and within the capsule receiving portion. In an embodiment, the at least one fixing element fixes the capsule and the implantable element as specified elsewhere herein. Thus, in an embodiment, the at least one fixing element is in direct contact with the capsule, with the implantable element, or with the capsule and the implantable element. However, as will be understood from the above, the at least one fixing element is not required to directly contact both the implantable element and the capsule for fixing both the implantable element and the capsule, since, e.g., fixing the capsule in cases where the capsule is distal from the implantable element may be sufficient; or by fixing the implantable element in cases where the implantable element is distal from the capsule, the capsule is fixed as well. In an embodiment, the at least one fixing element is in direct contact with the capsule. In a further embodiment, the capsule receiving portion or the implantable element receiving portion may comprise at least one further fixing element as specified herein.

In an embodiment, the at least one fixing element is a mechanical barrier, in an embodiment selected from the group consisting of a tongue, a crimp, a bulge, a spring element, and a taper. In an embodiment, the at least one fixing element is a tongue or bulge, e.g., produced by applying shearing forces, e.g., punched, from the material of the implantation needle; in an embodiment, said mechanical barrier fixes the capsule and/or the implantable element by elastically obstructing the inner lumen of the implantation needle, by exerting pressure on the capsule and/or the implantable element if inserted to their respective receiving portions, and/or fixing the capsule and/or the implantable element via a notch comprised therein. In a further embodiment, the mechanical barrier is a taper of the inner lumen of the implantation needle, which can be expanded upon exertion of external force. In a further embodiment, the mechanical barrier is a taper and the capsule is made of a soft or semisoft, i.e., deformable, material; as will be understood by the skilled person, in such case the implantable element, in an embodiment, has a diameter small enough to pass through the smallest diameter of the taper in such case.

In a further embodiment, the at least one fixing element is an adhesive surface. In an embodiment, the adhesive surface comprises, in an embodiment consists of, an adhesive which is shear thickening, biocompatible, and/or biodegradable. The term “adhesive” is known to the skilled person to relate to any compound capable of causing two objects to stick together. The term “shear thickening” is known to the skilled person to relate to a compound the viscosity of which increases with the rate of shear strain; thus, in an embodiment, the adhesive surface comprises, in an embodiment consists of, a dilatant adhesive. In an embodiment, the adhesive is slightly adhesive at room temperature, permitting movement of, e.g., the capsule with low force. Suitable adhesives are in principle known in the art, e.g., from Cohen et al. (2012), J Adhesion Science and Technology 27(18-19):1998, and include (i) elastomers, including natural rubbers, styrene block copolymers, acrylics, polyisobutylene and butyl rubbers, ethylene-vinyl acetate copolymers, and silicone elastomers, (ii) tackifier resins, including rosins and rosins derivatives, terpene resins, and hydrocarbon resins, and (iii) biodegradable/biobased elastomers, including epoxidized soybean oil, poly(ethylene citrate), gluten-based adhesives, wood adhesives, rapeseed oil adhesives, poly(hydroxyalkanoates), and poly(lactic acid)/polymenthide triblock polymers. In an embodiment, the adhesive is a biodegradable adhesive, in a further embodiment is poly(ethylene citrate). In an embodiment, the adhesive is a polymeric adhesive, in a further embodiment a non-crosslinked polymer. In an embodiment, the adhesive surface is included in the implantation needle in the production process of the implantation needle. It is, however, also envisaged that the adhesive surface is introduced into the implantation needle by a capsule and/or implantable element comprising said adhesive surface. In an embodiment, the adhesive of the adhesive surface is selected such that there is no chemical reaction with the capsule, i.e., there is neither a reaction with the pharmaceutical compound nor, in an embodiment, with the excipient.

The term “implantable element,” as used herein, relates to any physical object suitable for being implanted, partially or completely, into the body of a subject, which is not biodegradable and which is, in an embodiment, biocompatible. Implantable elements are, in principle, known in the art. In an embodiment, implantation (also referred to as “implanting”) is intra-tissue implantation, in an embodiment is subcutaneous implantation. In an embodiment, implanting of the implantable element and of the capsule into the body of a subject is simultaneous implantation, in a further embodiment simultaneous implantation with an implantation needle as specified herein. In an embodiment, the implantable element is partially implantable and is a device for administering a chemical compound, in an embodiment a liquid, into or out of the body of the subject. Thus, in an embodiment, the implantable element is a cannula, in particular an insulin cannula, a drainage tubing, an intravascular catheter, a pacemaker electrode, an oxygenator, an extracorporeal oxygenator tubing or accessory thereof, a dialyzer, a dialysis tubing or accessory thereof, a hemoadsorbent, or an immunoadsorbent. In a further embodiment, the implantable element is partially or completely implantable and is a sensor device, in particular a sensor adapted for determining the concentration of an analyte as specified herein below, in the body of a subject. In a further embodiment, the implantable element is a fully implantable sensor device. In an embodiment, the implantable element is a glucose sensor, in a further embodiment a continuous glucose sensor, in a further embodiment a fully implantable continuous glucose sensor. In an embodiment, the implantable element has an elongated or round form, in a further embodiment an elongated form, in an embodiment has an essentially round, rectangular, or trapezoidal cross-section, in particular has the form of a cylinder or a rounded cylinder. In an embodiment, the implantable element has an outer diameter of from 0.25 mm to 1.5 mm, in a further embodiment of from 0.5 mm to 1 mm, in a further embodiment of about 0.65 mm, in a further embodiment of 0.67 mm. As will be understood by the skilled person, the outer diameter of the implantable element is in an embodiment smaller than the inner diameter of the implantation needle. In an embodiment, the outer diameter of the implantable element is up to 80%, in a further embodiment up to 90%, in a further embodiment up to 95%, in a further embodiment up to 99% of the inner diameter of the implantation needle, in particular of the implantable element receiving portion. As will be further understood by the skilled person, the above applies mutatis mutandis to implantable elements having a cross-section profile with a limited number of symmetry axes, e.g., only one axis of symmetry, and to irregularly shaped implantable elements.

The term “analyte,” as used herein, relates to a chemical compound present in a liquid, in particular a bodily liquid. In an embodiment, the analyte is an organic molecule, in a further embodiment, an organic molecule capable of undergoing a redox reaction in the presence of the enzyme according to this disclosure. In an embodiment, the analyte is a molecule of a subject's metabolism, i.e., a molecule produced by and/or consumed in at least one chemical reaction taking place in at least one tissue of said subject. Also in an embodiment, the analyte is a low molecular weight chemical compound, in a further embodiment, a chemical compound with a molecular mass of less than 5000 u (5000 Da; 1 u=1.66×10⁻²⁷ kg), in a further embodiment, less than 1000 u, in a further embodiment, less than 500 u. I.e., in an embodiment, the analyte is not a biological macromolecule. In a further embodiment, the analyte is selected from the group consisting of glucose, malate, ethanol, ascorbic acid, cholesterol, glycerol, urea, 3-hydroxybutyrate, lactate, pyruvate, ketones, and creatinine; still in a further embodiment, the analyte is glucose.

The term “capsule,” as used herein, relates to an element comprising a solid or semisolid dosage form of at least one pharmaceutical compound, i.e., a pharmaceutically active ingredient comprised in the capsule. Thus, the capsule, in an embodiment, is a pellet, including a tablet, is a gel, or is a capsule sensu stricto. In an embodiment, the capsule is obtained or obtainable by granulating the pharmaceutical compound and, optionally, an excipient, and/or molding, in an embodiment hot-molding, or press-molding. In an embodiment, the capsule comprises at least one pharmaceutical compound and at least one excipient. In an embodiment, the capsule is or comprises a slow-release formulation of the pharmaceutical compound. Slow-release formulations are in principle known in the art, specific embodiments are specified herein below. In an embodiment, the capsule is a slow-release formulation releasing of from 10% to 50%, in an embodiment of from 15% to 40%, in a further embodiment of from 20% to 30% of the pharmaceutical compound within one week upon implantation into the body of a mammal. In an embodiment, the release kinetics of the capsule is essentially linear over at least one week, in an embodiment at least two weeks, in a further embodiment at least three weeks, in a further embodiment at least four weeks. The pharmaceutical compound may be formulated as a pharmaceutically acceptable salt. Pharmaceutically acceptable salts are in particular hydrochlorides, acetate salts, and phosphate salts.

Also, the pharmaceutical compound can be formulated in combination with other drugs either in a common pharmaceutical composition or as separated pharmaceutical compositions, wherein said separated pharmaceutical compositions may be provided in form of, e.g., a second capsule or a further layer of the capsule. In an embodiment, the capsule comprises of from 100 μg to 5 mg pharmaceutical compound, in a further embodiment of from 200 μg to 2 mg pharmaceutical compound, in a further embodiment of from 500 μg to 1.5 mg pharmaceutical compound. In an embodiment, the capsule comprises of from 100 μg to 5 mg excipient, in a further embodiment of from 150 μg to 2 mg excipient, in a further embodiment of from 200 μg to 1 mg, in a further embodiment about 400 μg excipient. In an embodiment, the capsule has an overall extended shape, in an embodiment the shape of a cylinder, a rod, or of a cone, in a further embodiment the shape of a cylinder. As is understood by the skilled person, the dimensions of the capsule are, in an embodiment, adapted to the dimensions of the inner lumen of the implantation needle, in particular the inner lumen of the section of the implantation needle from the capsule receiving section to the tip portion. In an embodiment, the capsule is cylindrical having a diameter of from 0.25 mm to 1.5 mm, in a further embodiment of from 0.5 mm to 1 mm, in a further embodiment of about 0.65 mm, in a further embodiment of 0.67 mm. In an embodiment, the capsule, in particular the cylindrical capsule having a diameter as specified above, has a length of from 0.5 mm to 5 mm, in a further embodiment of from 1 mm to 3 mm, in a further embodiment of about 1.8 mm, in a further embodiment of 1.8 mm.

In an embodiment, the pharmaceutical compound is a compound improving tolerance of an implantable element in the body of a subject. In an embodiment, improving tolerance is reducing immune rejection, reducing scarring, and/or reducing growth of infectious agents. Appropriate pharmaceutical compounds for improving tolerance are, in principle, known in the art. In an embodiment, improving tolerance is locally improving tolerance at the site of implantation, in an embodiment at the site of subcutaneous implantation. In an embodiment, the pharmaceutical compound is an immunosuppressive, anti-allergic and/or anti-inflammatory agent, in a further embodiment a glucocorticoid immunosuppressive agent, in a further embodiment is dexamethasone or betamethasone or a salt, derivative, and/or prodrug thereof, in a further embodiment is a mixture of dexamethasone and betamethasone. Betamethasone has the IUPAC-name (8 S,9R,10S,11S,13S,14S,16S,17R)-9-Fluoro-11,17-dihydroxy-17-(2-hydroxyacetyl)-10,13,16-trimethyl-6,7,8,11,12,14,15,16-octahydro-cyclopenta[a]phenanthren-3-one (CAS number 378-44-9). Dexamethasone has the IUPAC-name (8S,9R,10S,11S,13S,14S,16R,17R)-9-Fluoro-11,17-dihydroxy-17-(2-hydroxyacetyl)-10,13,16-trimethyl-6,7,8,11,12,14,15,16-octahydro-cyclopenta[a]phenanthren-3-one, (CAS number 50-02-2). In an embodiment, the derivative of Betamethasone or Dexamethasone is selected from the list consisting of 17-Oxo Betamethasone, Betamethasone hydrochloride, Betamethasone 21-acetate, Betamethasone 17-propionate, Betamethasone 21 propionate, Betamethasone phosphate, Betamethasone 17-valerate, Betamethasone 21-valerate, Betamethasone 17-benzoate, Betamethasone 17,21,-dipropionate, Betamethasone acibutate, Betamethasone sodium phosphate, Betamethasone butyrate propionate, Betamethasone tripropionate, 3-Hydroxy dexamethasone, 6 Hydroxydexamethasone, dexamethasone 17-acetate, dexamethasone 17-carboxamide, dexamethasone 17-propionate, dexamethasone 21-valerate, dexamethasone 21-acetate, dexamethasone 21-beta-D-glucoside, dexamethasone 21-linolate, dexamethasone 21-mesylate, dexamethasone 21-O-b-D-galactopyranose, dexamethasone 21-palmitate, dexamethasone 21-propionate, dexamethasone 21-sodium hydrogen phosphate, dexamethasone 21-sulfate, dexamethasone 21-tributylacetate, dexamethasone acefurate, dexamethasone 17-carboxylic acid, dexamethasone beta-D-glucuronide, dexamethasone dipropionate, Dexamethasone hemisuccinate, dexamethasone laurate, dexamethasone phosphate, dexamethasone sodium phosphate, dexamethasone tridecylate, dexamethasone valerate, dexamethasone 21-isonicotinate; in an embodiment is selected from the list consisting of Betamethasone 21-acetate, Betamethasone 17-propionate, Betamethasone 21 propionate, Betamethasone 17-valerate, Betamethasone 21-valerate, Betamethasone butyrate propionate, Betamethasone tripropionate, dexamethasone 17-acetate, dexamethasone 17-carboxamide, dexamethasone 17-propionate, dexamethasone 21-valerate, dexamethasone 21-acetate, dexamethasone 21-linolate, dexamethasone 21-mesylate, dexamethasone 21-palmitate, dexamethasone 21-propionate, dexamethasone 21-tributylacetate, dexamethasone acefurate, dexamethasone dipropionate, dexamethasone laurate, dexamethasone tridecylate, dexamethasone valerate. In an embodiment, the capsule comprises further pharmaceutically active compounds, in particular further anti-inflammatory, anti-allergic, and/or immunosuppressive compounds.

The term “excipient” is known to the skilled person to relate to a pharmaceutically inactive carrier compound in a pharmaceutical formulation being compatible with the other ingredients of the formulation and being not deleterious to the recipient thereof. In an embodiment, the excipient is biocompatible as specified herein above. In an embodiment, the excipient is biodegradable as specified herein above. In a further embodiment, the excipient is biocompatible and biodegradable. In an embodiment, the excipient comprises, preferably consists of, a polylactide, a polyglycolide, a polydioxanone, and/or a polyhydroxybutyrate. In an embodiment, the excipient comprises, preferably consists of, poly[(R)-3-hydroxybutyric acid], in an embodiment with an Mw of from 2500 to 200000; and/or poly(3-hydroxybutyric acid-co-3-hydroxyvaleric acid, in an embodiment with a mass ratio of 3-hydroxyvaleric acid of from 0% (w/w) to 100% (w/w), in an embodiment with an M_{w} of from 2500 to 200000, with M_{w} of the polymeric excipients as specified herein in an embodiment being determined by GPC according to van Dijk et al. (1983), Journal of Polymer Science: Polymer Chemistry Edition 21(1):197-208. In an embodiment, the excipient is selected from the group consisting of (i) polylactides, (ii) polyglycolides, (iii) polydioxanones, (iv) combinations of (i) to (ii), and (v) copolymers of monomers of at least two of (i) to (ii). In a further embodiment, the excipient comprises, in an embodiment consists of, a polylactide, a polyglycolide, or a combination of a polylactide and a polyglycolide. As is understood by the skilled person, the term “combination” in the context of the excipients of this disclosure includes mixtures as well as non-mixed combinations, such as layered combinations. The term “copolymers” includes statistical as well as block copolymers. In an embodiment, the excipient comprises, in an embodiment consists of, a polylactide or a poly(lactide-co-glycolide), in a further embodiment a poly(lactide-co-glycolide) 50/50.

The term “polylactide” relates to a polymer based on lactic acid or a derivative thereof as a monomer. Polylactides are polyesters and are also referred to as polylactic acids. In an embodiment, the polylactide is poly(lactic acid), CAS number 26100-51-6. In an embodiment, the polylactide is a polylactic acid with an M_{w} of from 2500 to 200000, in an embodiment with ester and/or acid end groups. The term “polyglycolide” relates to a polymer based on glycolic acid or a derivative thereof as a monomer. In an embodiment, the polyglycolide is poly(D,L-lactide-co-glycolide) 50:50, in an embodiment with an M_{w} of from 7000 to 69000 kDa, in an embodiment with ester and/or acid end groups. In an embodiment, the polyglycolide is poly(glycolic acid), CAS number 26009-03-0. The term “polydioxanone” relates to a polymer based on p-dioxanone or a derivative thereof as a monomer. In an embodiment, the polydioxanone is poly(p-dioxanone), CAS number 31621-87-1.

Advantageously, it was found in the work underlying this disclosure that tolerance-improving pharmaceutical compounds can be administered simultaneously to an implantable element without having to be physically attached to the implantable element, which increases flexibility with regard to the pharmaceutical compound administered and simplifies production of the implantable element. The implantation needle as described improves administration of the implantable element and the capsule; moreover, the capsule and the implantable element can be implanted in one step, improving compliance by the user.

The definitions made above apply mutatis mutandis to the following. Additional definitions and explanations made further below also apply for all embodiments described in this specification mutatis mutandis.

This disclosure further relates to an implantation kit for implanting an implantable element, said implantation kit comprising the implantation needle according to this disclosure, an implantable element comprised in the implantable element receiving portion of said implantation needle, and a capsule comprised in the capsule receiving portion of said implantation needle.

As used herein, the term “implantation kit” generally refers to a combination of components as specified, adapted for inserting the implantable element and the capsule into a body tissue, wherein the implantable element and the capsule, in an embodiment, are inserted simultaneously. Kits for subcutaneously inserting devices are in principle known to the skilled person, e.g., from WO 2016/012482 and references cited therein. The implantation kit as specified herein comprises an implantation needle as specified herein, such as according to any one of the embodiments listed above or listed in further detail below, at least one implantable element and at least one capsule, both as specified herein above. In the implantation kit, the implantable element is comprised in the implantable element receiving section and the capsule is comprised in the capsule receiving section, in order to be implanted into the body tissue, in an embodiment via a translational movement, in a further embodiment a common translational movement of the implantable element and the capsule, through the inner lumen of the implantation needle. Thus, in an embodiment, the components of the implantation kit, in particular the implantation needle, the implantable element, and the capsule are comprised in a common casing, optionally comprising further components of the implantation kit as specified herein functionally connected thereto. In an embodiment, all components required for subcutaneous insertion of the implantable element and the capsule are comprised in the casing of the implantation kit, i.e., in an embodiment, the implantation kit is a ready-to-use implantation kit. In an embodiment, thus, the implantation kit is a standalone system comprised of the components specified herein. In an embodiment, at least the implantation needle, the implantable element, and the capsule are sterile, in a further embodiment, the implantation kit is provided in a sterilized manner.

In an embodiment, the implantation kit further comprises a discharging unit adapted for discharging said implantable element and said capsule through said implantation needle, in an embodiment a plunger. The discharging unit may be driven manually by the user, e.g., by exerting longitudinal force on a plunger pushing the implantable element and the capsule in the direction of the tip portion of the implantation needle and into the tissue of the subject. In an embodiment, the discharging unit and, optionally the implantation needle itself, are functionally connected to a drive mechanism. Thus in an embodiment, discharging the implantable element and capsule is effected by a drive mechanism; in a further embodiment, discharging the implantable element and capsule as well as subcutaneous insertion of the implantation needle are effected by a drive mechanism. As will be appreciated, the implantation needle optionally may be retracted after discharging, in an embodiment by still the same drive mechanism. Moreover, the implantation kit, in an embodiment, further comprises an actuator, in an embodiment a user-operated actuator. The actual insertion process may be initiated by pressing at least one actuator arm or button by the user. By the drive mechanism, such as by transforming a motion of the actuator arm via a rotational movement of the rotator into a linear movement, the implantation needle with the implantable element and the capsule disposed therein may be driven. Thereby, the implantable element and the capsule may transcutaneously be inserted into the body tissue. Once the implantable element and capsule are inserted, the implantation needle may be pulled back. The implantation needle may be retracted into the casing of the implantation kit and may be secured therein. As will be understood by the skilled person, a drive mechanism may, in principle, also be driven by a spring element or an electric motor element; in an embodiment, the implantation kit further comprises a trigger in such case, said trigger eliciting release of the spring element or activating the electric motor element.

In an embodiment, the drive mechanism comprises a gear transmission or gearing. Still, additionally or alternatively, the drive mechanism may comprise other types of drives, such as one or more of a belt drive, a friction gear, a spindle drive, a spindle-top drive or combinations thereof. Other types of drive mechanisms are feasible. The actuator may be operable at a minimum force. The user simply has to apply sufficient force for actually transferring the implantation needle through the skin into the body tissue and for overcoming the restrain by the at least one fixing element. Thus, generally, the actuator and the drive mechanism in the present implantation kit may be designed such that delivery of the implantable element and the capsule, as well as optionally a piercing of the skin by the implantation needle and retraction of the implantation needle from the body tissue may be performed during one smooth actuation movement of the actuator arm or actuator arms, i.e., during one actuation process.

The implantation kit further may comprise at least one body patch adapted for attachment to a skin surface, such as to a skin surface of a subject, in particular in cases where the implantable element is not fully implantable. In order to be attached to the skin surface, the body patch specifically may comprise one or more adhesive patches and/or plasters and/or other types of attachment elements for attachment of the body patch to the skin surface. As will be understood by the skilled person, the adhesive in the aforesaid adhesive patch need not be the adhesive of the at least one fixing element as specified elsewhere herein and, in an embodiment, is a different adhesive. The body patch may be adapted to be coupled to the implantation kit during inserting the implantable element and capsule into the body tissue. Thus, the body patch may contain one or more receptacles adapted for receiving a portion of the implantation kit, in particular of the implantation needle and for holding the implantation needle.

This disclosure also relates to a method for administering an implantable element and a capsule to a subject, said method comprising inserting an implantation needle of an implantation kit according to this disclosure into a tissue of a subject and discharging said implantable element and said capsule into said tissue.

The method of this disclosure, in an embodiment, is an in vivo method. Moreover, it may comprise steps in addition to those explicitly mentioned above. For example, further steps may relate, e.g., to disinfecting the site of piercing by the implantation needle before insertion, and/or withdrawing said implantation needle after discharging. As is understood by the skilled person, subcutaneous implantation of suitable implantable element and capsules does not require specific medical skills and does not impose a significant health risk on a subject and, thus, is usually performed by the subject receiving said implantable element and capsule itself. Thus in an embodiment, the method is a method of self-administration.

This disclosure further relates to a use of an implantation needle according to this disclosure and/or of an implantation kit according to this disclosure for administering an implantable element and a capsule to a subject.

In view of the above, the following embodiments are particularly envisaged:

### Embodiment 1

An implantation needle comprising an implantable element receiving portion, a capsule receiving portion, and a tip portion, wherein the capsule receiving portion comprises at least one fixing element selected from the group consisting of a mechanical barrier and an adhesive surface.

### Embodiment 2

The implantation needle of embodiment 1, wherein the implantation needle is an open implantation needle, preferably a slotted implantation needle.

### Embodiment 3

The implantation needle of embodiment 1 or 2, wherein said mechanical barrier is selected from the group consisting of a tongue, a crimp, a bulge, a spring element, and a taper.

### Embodiment 4

The implantation needle of embodiment 1 to 3, wherein said mechanical barrier is produced by punching.

### Embodiment 5

The implantation needle of any one of embodiments 1 to 4, wherein said adhesive surface comprises, in an embodiment consists of, an adhesive which is shear thickening, biocompatible, and/or biodegradable.

### Embodiment 6

An implantation kit comprising the implantation needle according to any one of embodiments 1 to 5, an implantable element comprised in the implantable element receiving portion of said implantation needle, and a capsule comprised in the capsule receiving portion of said implantation needle.

### Embodiment 7

The implantation kit of embodiment 6, wherein said capsule is or comprises a solid or semisolid dosage form of at least one pharmaceutical compound, in an embodiment a solid dosage form, in a further embodiment a slow-release dosage form, in a further embodiment a pellet.

### Embodiment 8

The implantation kit of embodiment 6 or 7, wherein said capsule comprises at least one pharmaceutical compound.

### Embodiment 9

The implantation kit of any one of embodiments 6 to 8, wherein said capsule further comprises at least one excipient, in an embodiment wherein said at least one excipient is a biodegradable polymer, in an embodiment is selected from the group consisting of (i) polylactides, (ii) polyglycolides, (iii) polydioxanones, (iv) combinations of at least two of (i) to (iii), and (v) copolymers of monomers of at least two of (i) to (iii).

### Embodiment 10

The implantation kit of embodiment 9, wherein said at least one excipient comprises, in an embodiment consists of, a polylactide or a poly(lactide-co-glycolide).

### Embodiment 11

The implantation kit of any one of embodiments 7 to 10, wherein said pharmaceutical compound is a compound improving tolerance of said implantable element in the body of a subject.

### Embodiment 12

The implantation kit of embodiment 11, wherein said improving tolerance is reducing immune rejection, reducing scarring, and/or reducing growth of infectious agents.

### Embodiment 13

The implantation kit of embodiment 11 or 12, wherein said improving tolerance is locally improving tolerance at a site of implantation, in an embodiment subcutaneous implantation.

### Embodiment 14

The implantation kit of any one of embodiments 7 to 13, wherein said pharmaceutical compound is an immunosuppressive, anti-allergic and/or anti-inflammatory agent, in an embodiment a glucocorticoid immunosuppressive agent, in a further embodiment is dexamethasone or a derivative or prodrug thereof, in a further embodiment is dexamethasone.

### Embodiment 15

The implantation kit of any one of embodiments 6 to 14, wherein said implantable element is a sensor, in an embodiment a glucose sensor, in a further embodiment a fully implantable continuous glucose sensor.

### Embodiment 16

The implantation kit of any one of embodiments 6 to 14, wherein said implantable element is a cannula, in an embodiment an insulin delivery cannula.

### Embodiment 17

The implantation kit of any one of embodiments 13 to 16, wherein implantation is intra-tissue implantation, in an embodiment is subcutaneous implantation.

### Embodiment 18

The implantation kit of any one of embodiments 13 to 17, wherein implantation of said implantable element and said capsule into the body of a subject is simultaneous implantation.

### Embodiment 19

The implantation kit of any one of embodiments 6 to 18, wherein said implantable element and said capsule are configured for simultaneous implantation via said implantation needle.

### Embodiment 20

The implantation kit of any one of embodiments 6 to 19, wherein said implantation kit further comprises a discharging unit adapted for discharging said implantable element and said capsule through said implantation needle, in an embodiment the discharging unit is a plunger.

### Embodiment 21

The implantation kit of embodiment 20, wherein said implantation kit further comprises an actuator and/or a trigger adapted for effecting discharging of said implantable element and said capsule.

### Embodiment 22

The implantation kit of any one of embodiments 19 to 21, wherein said implantable element and said capsule are preconfigured in said implantation kit for discharge through said implantation needle.

### Embodiment 23

The implantation kit of any one of embodiments 6 to 22, wherein said implantation kit is a ready-to-use implantation kit.

### Embodiment 24

A method for administering an implantable element and a capsule to a subject, said method comprising inserting an implantation needle of an implantation kit according to any one of embodiments 6 to 23 into a tissue of a subject and discharging said implantable element and said capsule into said tissue.

### Embodiment 25

The method of embodiment 24, wherein said method is a method of self-administration.

### Embodiment 26

Use of an implantation needle according to any one of embodiments 1 to 5 and/or of an implantation kit according to any one of embodiments 6 to 23 for administering an implantable element and a capsule to a subject.

### Embodiment 27

The method of embodiment 24 or 25 or the use of embodiment 26, wherein said administering is implantation, in an embodiment subcutaneous implantation.

All references cited in this specification are herewith incorporated by reference with respect to their entire disclosure content and the disclosure content specifically mentioned in this specification.

### BRIEF DESCRIPTION OF THE DRAWINGS

The above-mentioned aspects of exemplary embodiments will become more apparent and will be better understood by reference to the following description of the embodiments taken in conjunction with the accompanying drawings, wherein:

FIG. 1 shows a top view of an exemplary embodiment of the implantation kit of this disclosure;

FIG. 2 shows a front view of the exemplary embodiment of FIG. 1; and

FIG. 3 shows a cross-sectional view of the exemplary embodiment of FIG. 1 along axis A-A′.

### DESCRIPTION

The embodiments described below are not intended to be exhaustive or to limit the invention to the precise forms disclosed in the following detailed description. Rather, the embodiments are chosen and described so that others skilled in the art may appreciate and understand the principles and practices of this disclosure.

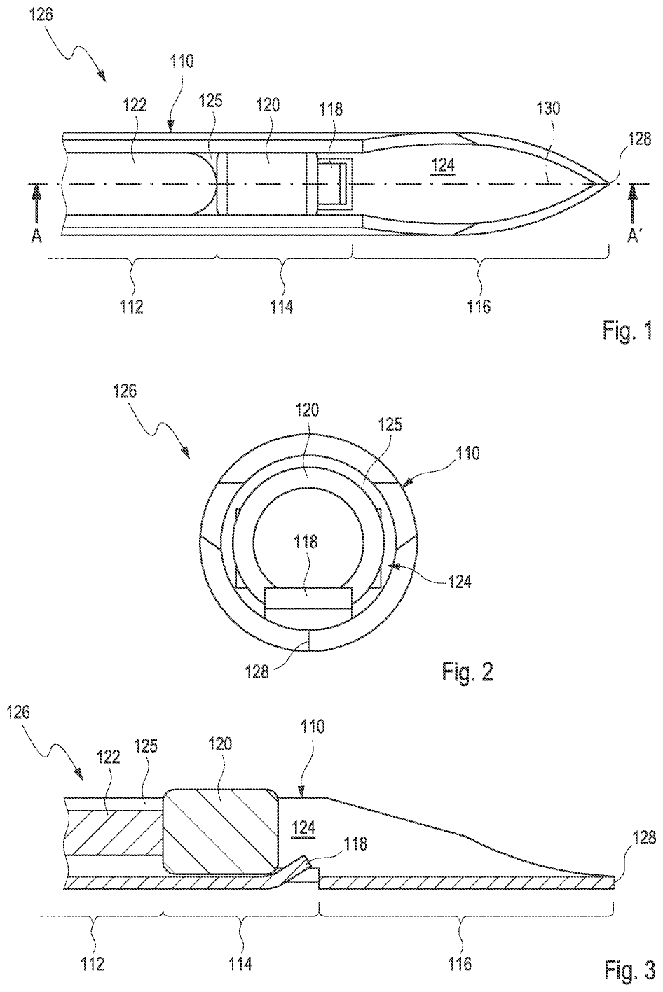

In FIGS. 1 to 3, an exemplary embodiment of an implantation needle 110 and an implantation kit 126 is shown in various views. Therein, FIG. 1 shows a top view, FIG. 2 shows a front view, and FIG. 3 shows a cross-sectional view through the implantation needle 110 along axis A-A′ (denoted by reference number 130) in FIG. 1. As shown, e.g., in FIG. 1 or 3, the implantation kit 126 comprises the implantation needle 110, at least one implantable element 122 and at least one capsule 120.

The implantation needle 110 has a generally open needle body and a plurality of portions, which are shown in FIG. 1. Thus, in this embodiment, the implantation needle 110 comprises, e.g., in the given order in a direction towards a tip 128 of the implantation needle 110, the following portions (also referred to herein as “sections”):
at least one an implantable element receiving portion 112,
at least one capsule receiving portion 114, and
at least one tip portion 116.

In the implantation kit 126 as shown in the Figures, the at least one implantable element 122 is fully or at least partially received in the at least one implantable element receiving portion 112, and the capsule 120 is fully or at least partially received in the at least one capsule receiving portion 114. The tip portion 116, as an example, may be empty. The implantable element 122 and the capsule 120 may fully be received in an inner lumen 124 of the implantation needle 110 or each may also slightly protrude through slot 125, such as shown for the capsule 120 in FIG. 3. Still, even when protruding from the slot 125, the dimensions and the geometries of the implantable element 122, the capsule 120 and the slot 125 may be chosen such that the implantable element 122 and the capsule 120 may not leave the inner lumen 124 in a direction perpendicular to longitudinal axis 130 and may only slide in a direction parallel to axis 130.

The portions 112, 114, 116 may be portions of the implantation needle 110, which, may, e.g., be embodied as a slotted implantation needle. The implantation needle 110 may, e.g., be punched from a sheet of appropriate material, such as a metal, and/or be embossed to the form as shown. The implantation needle 110 may, however, also be cylindrically closed. The portions may be separated by separating elements, such as protrusions or a tip of the fixing element 118 described in further detail below, or may simply be separated by virtual lines. In the portions, the implantation needle 110 may have identical diameters or equivalent diameters. Alternatively, the diameters or equivalent diameters of the portions may differ.

In the capsule receiving portion 114, the implantation needle 110 comprises a fixing element 118 for fixing the capsule 120 and for preventing the capsule 120 from unwanted sliding towards the tip 128. The fixing element 118, as an example, may be in the form of a tongue, which may in particular be punched from the material of the implantation needle 110 and bent to a form obstructing the inner lumen of the implantation needle 110, which is shown in the front view of FIG. 2 or in the cross-sectional view of FIG. 3.

In the exemplary embodiment shown in the Figures, the order of the respective portions is implantable element receiving portion 112, capsule receiving portion 114, and tip portion 116. Thus, the at least one fixing element 118 restrains the capsule 120 from moving towards tip portion 116 in the absence of external force. Upon exertion of external force, in particular a force along axis A-A′ in the direction of tip portion 116, the tongue serving as fixing element 118 is forced downward in the configuration of FIGS. 2 and 3, thereby unblocking the inner lumen of implantation needle 110. Thus, the fixing element 118, as an example, may be embodied as a spring element and/or may have elastic properties. As shown in FIG. 2, the fixing element 118 may obstruct the lumen of the implantation needle 110, thereby preventing the capsule 120 and the implantable element 122 from moving in the direction of the tip portion 116. The fixing element 118, via the capsule 120, may further prevent the implantable element 122 from moving towards the tip 128. Thus, the fixing element 118 may prevent both the capsule 120 and the implantable element 122 from moving in the direction of tip portion 116.

The capsule 120 may be produced according to methods in principle known in the art (Meyer et al. (2012), Retina 32:2133; Fialho & da Silva Cunha (2005), Drug Delivery 12(2): 109). For an implantation period of about 30 days, the capsule 120 may have a cylindrical form with, e.g., a diameter of 0.67 mm and a length of about 1.8 mm.

While exemplary embodiments have been disclosed hereinabove, the present invention is not limited to the disclosed embodiments. Instead, this application is intended to cover any variations, uses, or adaptations of this disclosure using its general principles. Further, this application is intended to cover such departures from the present disclosure as come within known or customary practice in the art to which this invention pertains and which fall within the limits of the appended claims.

### LIST OF REFERENCE NUMBERS

110 implantation needle
112 implantable element receiving portion
114 capsule receiving portion
116 tip portion
118 fixing element
120 capsule
122 implantable element
124 inner lumen of implantation needle
125 slot
126 implantation kit
128 tip
130 axis

What is claimed is:

1. An implantation needle, comprising:
an open needle body having a tip portion;
an implantable element receiving portion configured to receive an implantable element;
a capsule receiving portion configured to receive a capsule; and
a fixing element configured to retain the capsule within the capsule portion, the fixing element comprising one of a mechanical barrier and an adhesive surface.

2. The implantation needle of claim 1, wherein the fixing element comprises the mechanical barrier and the mechanical barrier is selected from the group consisting of a tongue, a crimp, a bulge, a spring element, and a taper.

3. The implantation needle of claim 1, wherein the fixing element comprises the adhesive and the adhesive is shear thickening, biocompatible, and/or biodegradable.

4. An implantation kit, comprising:
the implantation needle of claim 1;
an implantable element disposed in the implantable element receiving portion; and
a capsule disposed in the capsule receiving portion.

5. The implantation kit of claim 4, wherein the capsule comprises a pharmaceutical compound.

6. The implantation kit of claim 5, wherein the capsule further comprises a biodegradable polymer excipient.

7. The implantation kit of claim 5, wherein the pharmaceutical compound comprises a compound configured to improve tolerance of the implantable element in the body of a subject.

8. The implantation kit of claim 5, wherein the pharmaceutical compound comprises an immunosuppressive, anti-allergic, and/or anti-inflammatory agent.

9. The implantation kit of claim 5, wherein the pharmaceutical compound comprises dexamethasone or a derivative thereof.

10. The implantation kit of claim 4, wherein the implantable element is a sensor or a cannula.

11. The implantation kit of claim 4, wherein the implantation needle is configured for simultaneous implantation of the implantable element and the capsule into the body of a subject.

12. A method for administering an implantable element and a capsule to a subject, said method comprising:
providing an implantation kit according to claim 4;
inserting the implantation needle into a tissue of a subject and discharging the implantable element and the capsule into the tissue.

13. The method of claim 12, wherein the method is a method of self-administration.
